(19) United States
(12) Patent Application Publication
(10) Pub. No.: US 2023/0321195 A1
(43) Pub. Date: Oct. 12, 2023

(54) EXOSOME ENCODED IL-10 AS A TREATMENT FOR INFLAMMATION ASSOCIATED ADVERSE PREGNANCY CONDITION

(71) Applicant: Board of Regents, The University of Texas System, Austin, TX (US)

(72) Inventors: Ramkumar Menon, Galveston, TX (US); Ananth Kumar Kammala, Galveston, TX (US); Enkhtuya Radnaa, Galveston, TX (US)

(21) Appl. No.: 18/296,755

(22) Filed: Apr. 6, 2023

Related U.S. Application Data
(60) Provisional application No. 63/329,125, filed on Apr. 8, 2022.

(51) Int. Cl. A61K 38/20; A61P 15/06

(52) U.S. Cl. CPC A61K 38/2066; A61P 15/06

(57) ABSTRACT

The present invention includes compositions and methods of treating a pregnant female subject at risk of preterm birth, comprising administering to the pregnant female subject an effective amount of an exosome that comprises a nucleic acid that expresses IL-10 or that comprises IL-10, wherein the effective amount of the IL-10 is effective to reduce, prevent or delay preterm birth

### CROSS-REFERENCE TO RELATED APPLICATIONS

This application claims priority to U.S. Provisional Application Ser. No. 63/329,125, filed Apr. 8, 2022 entitled “Exosome Encoded IL-10 As a Treatment for Inflammation Associated Adverse Pregnancy Condition”, which is hereby incorporated by reference in its entirety.

### TECHNICAL FIELD OF THE INVENTION

The present invention relates in general to the field of treatments for pregnancy associated conditions, and more particularly, to the use of IL-10 encoded in exosomes for the treatment of inflammation-associated adverse pregnancy conditions like spontaneous preterm birth and preterm premature rupture of the membranes.

### STATEMENT OF FEDERALLY FUNDED RESEARCH

Not applicable.

### INCORPORATION-BY-REFERENCE OF MATERIALS FILED ON COMPACT DISC

None.

### BACKGROUND OF THE INVENTION

Without limiting the scope of the invention, its background is described in connection with inflammation-associated adverse pregnancy conditions.

Preterm birth (PTB; birth before 37 weeks gestation) impact ˜11% of all pregnancies contributing to neonatal morbidity and mortality'.

One such treatment is described in U.S. Pat. No. 11,197,868, issued to Lockwood, et al., entitled “Prevention of preterm birth (PTB) by inhibition of FKBP51”. These inventors are said to teach a method of enhancing progesterone receptor (PR) activity in a mammal that comprises administering a composition comprising an inhibitor of FK506 binding protein 51 (FKBP51) to a mammal in need thereof, such as a pregnant human female, whereby progesterone receptor activity in the mammal is enhanced when compared to a mammal not administered the composition. It is further said that the method results in an extension of the gestation period and a decreased likelihood of preterm birth and fetal growth restriction.

Another such treatment is described in U.S. Pat. No. 11,154,562 and 10,471,075, issued to Birch, et al., are both entitled “Methods of reducing risk of preterm birth.” These inventors are said to teach methods for reducing the risk of preterm birth in a pregnant human female patient that include subcutaneous administration of 17-alpha-hydroxyprogesterone caproate (17-HPC or HPC). These inventors teach that the subcutaneous administration of HPC can result in sufficient plasma levels of HPC in pregnant patients that can reduce the risk of preterm birth. However, due to lack of mechanistic evidence and changes in progesterone levels are not one of the causal factors, these therapies have not been successful in reducing preterm birth risk.

Current drugs in use do not cross the feto-maternal (F-M) interface and thus cannot treat the fetal side contributing to increased neonatal mortality and morbidity. Drugs that cross the F-M interface either have teratogenicity or show no benefits to the neonates. Mechanistically, inflammation at the F-M interface is one of the key triggers of delivery both at term (physiologic) and preterm (pathologic). Current PTB prevention strategies prolong labor by minimizing contractions of maternal uterine tissues or prolong cervical remodeling to provide enough time for clinicians to administer corticosteroids that will benefit lung maturation (to avoid bronchopulmonary dysplasia, a major neonatal complication of PTB).

Thus, current drugs do not directly address inflammation, specifically the fetal inflammatory response associated with parturition. Multiple drugs (e.g., tocolytics, progesterone) have been tested; however, none have reduced the risk of PTB as indicated by the sustained rate of PTB in the past 30 years¹. All drugs in use are functional at the maternal uterine tissues (cervix and myometrium) and hardly reach the fetus, which plays an important role in triggering parturition. Therefore, fetal responses that result in PTB are not addressed when treating women having preterm labor. This is especially true in cases of infection and oxidative stress (OS, non-infectious)-associated PTB where fetal inflammatory response is a key trigger for preterm labor.³⁻⁶

Despite these advances, the current interventions do not address “fetus as a patient” in utero as most drugs either do not cross placental barriers or if they do cross, they are teratogenic.

### SUMMARY OF THE INVENTION

As embodied and broadly described herein, an aspect of the present disclosure relates to a method of treating a pregnant female subject at risk of preterm birth, comprising administering to the pregnant female subject an effective amount of an exosome that comprises a nucleic acid that expresses IL-10 or that comprises IL-10, wherein the effective amount of the IL-10 is effective to reduce, prevent or delay preterm birth. In one aspect, the preterm birth is spontaneous preterm birth. In another aspect, the pregnant human female presents to labor and delivery with symptoms of preterm birth. In another aspect, the female subject is a pregnant human at 20 to 37 weeks of gestation. In another aspect, the pregnant human female subject is at risk of preterm birth. In another aspect, the pregnant human female subject at risk of preterm birth has a short cervical length, an infection, a placental anomaly, has had a prior cesarean delivery, has or had uterine fibroids, a connective tissue disorder, is pregnant from in vitro fertilization, diabetes, blood clotting problems, high blood pressure, vaginal bleeding, a personal history of preterm birth, a family history of preterm birth, a previous pregnancy with 18 months of the current pregnancy, a current multi-fetal pregnancy, a uterine abnormality, a cervical abnormality, is overweight before or during pregnancy, is underweight before or during pregnancy, or is carrying a fetus with known birth defects. In another aspect, the pregnant human female subject at risk of preterm birth is a human woman under the age of 20 or over the age of 35. In another aspect, the pregnant human female subject at risk of preterm birth is a human woman engaging in smoking, engaging in drinking alcohol, using illegal drugs, with limited or no healthcare during pregnancy, subject to stress, subject to long working hours with long periods of standing, or exposed to environmental pollutants. In another aspect, the method further comprises checking the female subject for signs of preterm labor. In another aspect, the method further comprises checking the female subject for signs of preterm labor comprises a cervical exam, a transvaginal ultrasound exam, testing for amniotic fluid, or testing for fetal fibronectin. In another aspect, the exosomes that expresses IL-10 or that comprises IL-10 are not administered intraamnionically. In another aspect, the exosomes that expresses IL-10 or that comprises IL-10 are administered via a route that is oral, parenteral, intravenous, intra-arterial, subcutaneous, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, via inhalation, or combinations thereof.

As embodied and broadly described herein, an aspect of the present disclosure relates to a method of treating a pregnant female subject at risk of preterm birth, comprising administering to the pregnant female subject an effective amount of an exosome that comprises a nucleic acid that comprises IL-10 (eIL-10), wherein the effective amount of the eIL-10 is effective to reduce, prevent or delay preterm birth. In one aspect, the preterm birth is spontaneous preterm birth. In another aspect, the pregnant human female presents to labor and delivery with symptoms of preterm birth. In another aspect, the female subject is a pregnant human at 20 to 37 weeks of gestation; wherein the pregnant human female subject is at risk of preterm birth; or wherein the pregnant human female subject at risk of preterm birth has a short cervical length, an infection, a placental anomaly, has had a prior cesarean delivery, has or had uterine fibroids, a connective tissue disorder, is pregnant from in vitro fertilization, diabetes, blood clotting problems, high blood pressure, vaginal bleeding, a personal history of preterm birth, a family history of preterm birth, a previous pregnancy with 18 months of the current pregnancy, a current multi-fetal pregnancy, a uterine abnormality, a cervical abnormality, is overweight before or during pregnancy, is underweight before or during pregnancy, or is carrying a fetus with known birth defects. In another aspect, the pregnant human female subject at risk of preterm birth is a human woman under the age of 20 or over the age of 35. In another aspect, the pregnant human female subject at risk of preterm birth is a human woman engaging in smoking, engaging in drinking alcohol, using illegal drugs, with limited or no healthcare during pregnancy, subject to stress, subject to long working hours with long periods of standing, or exposed to environmental pollutants. In another aspect, the method further comprises checking the female subject for signs of preterm labor. In another aspect, the step of checking the female subject for signs of preterm labor comprises a cervical exam, a transvaginal ultrasound exam, testing for amniotic fluid, or testing for fetal fibronectin. In another aspect, the exosomes that eIL-10 is not administered intraamnionically.

As embodied and broadly described herein, an aspect of the present disclosure relates to an exosome comprising a nucleic acid that expresses IL-10 or that comprises IL-10, wherein the exosomes are isolated for size and loading of the nucleic acid that expresses IL-10 or the IL-10. In one aspect, the exosomes are isolated from a media by at least one of: ultracentrifugation, purification, or size exclusion chromatography. In another aspect, the exosomes have a particle size ranging between 40 nm-110 nm. In another aspect, the nucleic acid that expresses IL-10 or the IL-10 are loaded into the exosomes by one or more electroporation steps. In another aspect, the exosomes are autologous.

### BRIEF DESCRIPTION OF THE DRAWINGS

For a more complete understanding of the features and advantages of the present invention, reference is now made to the detailed description of the invention along with the accompanying figures and in which:

FIGS. 1A to 1H: Properties of naïve EVs vs EVs encapsulating IL-10. FIG. 1A. Nanoparticle Tracking Analysis using ZetaView showing EV size in nanometers prior to electroporation. FIG. 1B. Optimization of electroporation protocol. IL-10 was encapsulated in EVs via sonication or electroporation at 500 Volts with either 2 or 6 pulses. Six pulses at 500 V were optimum and provided the highest loading of IL-10 within EVs. These conditions were used for the generation of eIL10 in further experiments. FIG. 1C. Cryo EM images showing the morphology of EVs. Representative images of naïve and electroporated EVs show similar morphology. Both EVs were round with double layered membranes. FIG. 1D. Nanoparticle Tracking Analysis using ZetaView showing EV size in nanometers after electroporation. The particles do not show a significant change in size after electroporation compared to nonelectroporated EVs (1A). FIG. 1E. ExoView analysis of EV markers: CD81 (red), CD63, and CD9 (green) were determined by ExoView and confirmed the presence of EV markers that are not altered by electroporation or encapsulation of IL-10. FIG. 1F. Confirmation of IL-10 loading in EVs. Recombinant (r) IL-10 was conjugated with FITC and its presence in EVs was determined by fluorescence under UV light. Naïve EVs and EVs that were electroporated with only 2 pulses do not show fluorescence. EVs electroporated with 6 pulses, or EVs mixed with IL-10 without electroporation show higher levels of fluorescence. FIG. 1G. Confirmation of IL-10 loading in EVs using Exoview analysis. EVs captured to ExoView chips using tetraspanin markers were probed with IL-10 antibody to detect its luminal presence. FIG. 1H. Compared to naïve exosomes (white), eIL-10 EVs (blue) show presence of IL-10, and loading efficacy was calculated up to 70%. Data are shown as means ±SEM. For all groups, n≥5. Data are shown as means ±SEM. Data were analyzed using a one-way ANOVA with a Tukey's post hoc test. ***P≤0.001.

FIGS. 2A to 2F: Activity of eIL10 at IL-10 pathway and cytokine production. FIG. 2A. Schematic of secreted embryonic alkaline phosphatase (SEAP) reporter assay to determine the functional properties of IL-10 in EVs. HEK cells transfected with SEAP reporter where IL-10/IL-10 receptor binding leads to signaling activation and causing a color change. The color change can be measured by optical density. FIG. 2B. Optical density (OD) measured in SEAP-transfected cells. Naïve EVs does not contain IL-10. eIL10 shows binding to the receptor in a dose-dependent manner, contributing to SEAP activation as detected by increased OD. FIG. 2C. Determining the impact of eIL-10 on cell cycle using flow cytometry. Cell cycle analysis was performed in RAW264.7 (mouse macrophage) cells treated with naïve EVs or eIL10. Flow cytometry showed no significant changes in the number of cells found in each stage of the cell cycle. FIG. 2D. Determining the impact of eIL-10 on cell fate using flow cytometry. Analysis of apoptosis and necrosis using Annexin and propidium iodide staining in RAW cells treated with naïve EVs or eIL10. Neither naïve nor eIL-10 produced any necrosis of cells. Similarly, apoptotic cell deaths were <1% in both naïve and eIL-10 treated cells. FIG. 2E. Functional properties of eIL-10. ELISA was used to measure the levels of inflammatory cytokines produced by RAW264.7 cells (Mouse macrophages) treated with LPS and co-treated with either eIL10 or rIL10. LPS (red)-induced IL-6, TNF-a, and IL-1b co-treatment with eIL-10 (blue) rIL-10 (green) reduced the production of pro-inflammatory cytokines confirming functionally viable IL-10 inside the EVs. FIG. 2F. Percentage of apoptotic cells naïve EVs v eIL-10. For all groups, n≥5. Data are shown as means±SEM. Data were analyzed using a one-way ANOVA with a Tukey's post hoc test. *P≤0.05, **P≤0.01, ***P≤0.001, ****P≤0.0001.

FIGS. 3A to 3F: Bioavailability and effect of eIL10 in LPS-induced PTB mouse model. FIG. 3A. Left panel: Representative In vivo imaging system (IVIS) images. Fluorescently labeled IL-10 is seen in the placenta and fetal membrane when it is packaged in the EVs (FITC eIL-10) but not when rIL-10 was injected directly (FITC rIL-10). Controls remain negative, Right panel: Radiant efficiency in target tissues is shown for eIL10 (red) and rIL-10 (green). FIG. 3B. Schematic representation of LPS-(100 μg) (intraperitoneally injected) induced PTB model and its treatment using eIL-10 (500 ng) (intravenously injected through the tail vain). FIG. 3C. Survival curve comparing PBS (black), LPS (red) and LPS+eIL-10 (blue) treatment groups. LPS injections caused PTB within 24 hours, while LPS+eIL-10 delayed PTB by an average of 24 hours, compared to controls. For all groups, n≥8. FIG. 3D. Bar graph of number of hours of pregnancy maintained after eIL-10 treatment. eIL-10 delays PTB in LPS-challenged pregnant CD-1 mice. FIG. 3E. Representative western blot images of fetal membranes collected 6 hours after treatment with LPS and eIL-10. eIL-10 treatment reduced NF-κB activation (reduction of Phosphorylated (p) NF-κB) compared to LPS. FIG. 3F. ELISA of maternal plasma from blood collected 6 hours after treatment shows a decrease in IL-6 in mice treated with eIL10 compared to LPS. For all groups, n≥5. Data are shown as means±SEM. Data were analyzed using a one-way ANOVA with a Tukey's post hoc test. *P≤0.05, **P≤0.01, ***P≤0.001, ****P≤0.0001.

FIGS. 4A to 4E: Effect of eIL10 in E. coli-induced ascending infection mouse model. FIG. 4A. Schematic representation of ascending infection-induced preterm birth mouse model and intravenous treatment with eIL10 (500 ng) and/or gentamicin (10 mg/kg). Infection is induced by vaginal administration of E. coli (1010 colony forming units [CFU]). FIG. 4B. Survival graph comparing liquid broth, LB (black), E. coli (red), E. coli+eIL-10 (blue), E. coli+gentamicin (green), E. coli+eIL-10+gentamicin (purple) and only eIL-10 (pink) treatment groups. E. coli treatment caused PTB within 48 hours, while E. coli+eIL-10 delayed PTB to term delivery with live pups whereas gentamicin treatment delays only 24 hours compared to E. coli group. For all groups, n≥4. FIG. 4C. Delay in preterm birth (in hours) shown for each treatment group. Color key as seen in FIG. 4B. A significant delay in preterm birth is shown in mice treated with eIL10 or eIL10 and gentamicin compared to challenge with E. coli. FIG. 4D. Western blot of fetal membranes collected from pregnant mice 24 hours after treatment. Pregnant mice were treated with vehicle liquid broth (LB), E. coli, co-treatment of E. coli and eIL10, or eIL10 without E. coli challenge. E. coli-induced increase of activated NF-κB (phosphorylated: pNF-κB) is reversed with eIL10 administration. I-κB-α (an inhibitor of NF-κB is increased with treatment of eIL10. FIG. 4E. Western blot of fetal membranes showing levels of phosphorylated NF-κB normalized to total NF-κB and I-κB-α normalized to β-actin in fetal membranes collected from mice 24 hours after treatment as measured in western blot. Color key as seen in FIG. 4B. Immune challenge tests: Serum levels of IL-6 production measured by ELISA in pups born after exposure to E. coli, eIL10, and gentamicin treatment in utero. No significant changes are seen in IL-6 production in pups regardless of exposure indicative of lack of long term immune supression. Levels of TNF-α production in pups exposed to E. coli, eIL10, and gentamicin treatment in utero measured by ELISA. Color key as seen in FIG. 4B. No significant changes are seen in pups' TNF-α production. For all groups, n>5. Data are shown as means±SEM. Data were analyzed using a one-way ANOVA with a Tukey's post hoc test.

FIGS. 5A and 5B: eIL-10 inhibits NF-κB pathway. FIG. 5A. Representative western blot images of cell lysates collected from RAW264.7 (mouse macrophage) cells treated with LPS (red bar) and co-treated with either eIL10 (blue bar) or rIL10 (green bar) for a treatment time of 0, 15, or 30 minutes. LPS-induced increases of activated NF-κB (phosphorylated: p-NF-κB) and activated ERK (phosphorylated: p-ERK) were reversed by treatment with eIL10 or rIL10. Co-treatment of eIL10 or rIL10 also increased levels of I-κB-α (inhibitor of NF-κB) and phosphorylated STAT (p-STAT). Bar graphs show relative intensity of these bands compared to β-actin, and phosphorylated ERK shows relative intensity against total ERK. All the experiments were repeated three times. Normalized data were shown as means±SEM. Data were analyzed using a one-way ANOVA with a Tukey's post hoc test. *P≤0.05, **P≤0.01, ***P≤0.001, ****P≤0.0001. FIG. 5B. Immunocytochemistry staining of RAW264.7 cells with activated NF-κB (phosphorylated: pNF-κB) antibody (green) and DAPI (blue) to show translocation of p-NF-κB into the nucleus.

FIGS. 6A to 6D: eIL10 treatment reduced neutrophil infiltration and HCA. FIG. 6A. Schematic representation of a transgenic animal model with a membrane-targeted, 2-color fluorescent Cre-reporter allele where membrane-expressed tandem dimer Tomato (tdTomato—mT+). mT+males were mated with wild-type female (WT) mice, all fetal tissues had the mT/mG construct expressing mT+ (red fluorescence), keeping maternal tissues negative. FIG. 6B. Schematic representation of E. coli challenge and treatment of mice, various approaches of analysis of samples and data collection. Using the pregnant mouse model (FIG. 6A), mice are treated with vaginal administration of 104 colony forming units (CFU) of E. coli to induce ascending infection. Co-treatment with eIL10 is administered at a dose of 500 ng (intravenous through tail vein). FIG. 6C. Immunohistochemistry of fetal membranes 24 hours after administration of vehicle liquid broth (LB), E. coli challenge, co-treatment of E. coli with eIL10, or eIL10 without E. coli challenge. Tissues were stained with Ly-6G (green) for neutrophils and DAPI (blue) to show nuclei. FIG. 6D. Violin graphs show significant reduction in neutrophil levels by eIL10 (blue) after challenge with E. coli (red) as seen in IHC fluorescent microscopy. The number of neutrophils in fetal membranes is reduced to levels similar to those seen in vehicle liquid broth (LB, black) and eIL10 treatment alone (pink). For all groups, n≥5. Data are shown as means ±SEM. Data were analyzed using a one-way ANOVA with a Tukey's post hoc test. *P≤0.05, **P≤0.01, ***P≤0.001, ****P≤0.0001.

FIGS. 7A and 7B. Fetal specific Immune cell characterization during pregnancy in different treatment groups after 24 hours by mass cytometer. FIG. 7A. Identify differentially distributed cellular phenotypes by t-SNE in concatenated control, E. coli and E. coli+eIL-10 treated groups, respectively. FIG. 7B. The heatmaps shows the relative intensity of each parameter for different clusters in different groups. The percentage events of the particular cluster were determined by the bar charts from the cluster explorer tool and cluster was characterized by the expression profile expression markers. The characterized clusters percentage events were compared between different treatment groups. For all groups, n≥5. Data are shown as means±SEM. Data were analyzed using a one-way ANOVA with a Tukey's post hoc test. *P≤0.05, **P≤0.01, ***P≤0.001, ****P≤0.0001.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors have recognized that infection and inflammation are the key factors associated with preterm births (PTB)². In vitro studies and animal models have shown that anti-inflammatory interleukin (IL)-10 may reduce inflammation that is found in PTB. The requirement of invasive and often risky intraamniotic administration of IL-10 to reduce fetal inflammation has hindered its advancement in clinical trials.

The present inventors have developed a novel technology to engineer extracellular vesicles (EVs-exosomes of 40-175 nm) using electroporation approaches that can cross the blood-placental barrier to reduce the fetal inflammatory response. To advance this novel drug into clinical trials, the present inventors have determined their efficacy and mechanism of action (MOA). The objective was to test the mechanism of action of engineered EV encoded IL-10 in reducing fetal inflammation in response to a risk factor (e.g., in vitro and in vivo infection). The inventors developed an intervention strategy for PTB using EV (specifically exosomes) as a drug (IL-10) delivery vehicle and tested its efficacy in reducing fetal inflammatory responses in in vitro and in vivo models of infection/inflammation associated PTB.

Infectious or non-infectious inflammation contribute to over 70% of all PTB and neonatal morbidities. Current interventions are designed to delay PTB by reducing maternal uterine contractions. These approaches are not successful in reducing PTB rates (11% of all pregnancies), with a health care cost of ˜$26.5 billion/year in the United States alone to treat prematurity¹.

Inflammatory response at term and in PTB: Feto-maternal reproductive tissues maintain immune homeostasis during pregnancy and tolerate the semi-allogeneic fetus until parturition.⁷⁻¹² Balanced immune interactions by feto-maternal units (F-M, including fetal membrane, placenta, uterus, decidua, and cervix) ensure pregnancy maintenance and feto-placental growth.^{9,13,14} Pregnancy success is determined by regulatory mechanisms at the F-M tissues, ensuring that both the innate and adaptive immune cells aptly support feto-placental development by suppressing inflammation while remodeling uterine tissues.¹⁵⁻¹⁹ Parturition in both humans and animals is associated with a physiological inflammatory process.²⁰⁻²⁴ This inflammation is characterized by infiltration and activation of immune cells into the F-M units, along with an increased production of pro-inflammatory mediators and decreased levels of anti-inflammatory mediators.^{14,25-34} Disruption of immune homeostasis leading to parturition is expedited by both endocrine and paracrine mediators generated^{35,36} when fetal growth is complete.^{21,37-43} Premature disruption of immune homeostasis and overwhelming host inflammatory response due to infectious or other non-infectious risk factors can lead to PTB.^{22,27,44} Fetal inflammatory response is a key event in PTB. For example, ongoing studies by the present inventors have determined that lipopolysaccharide (LPS) and OS-induced PTB in mice is associated with an influx of fetal innate immune cells, not maternal, into feto-maternal uterine tissues along with proinflammatory NF-kB activation.

Interleukin (IL)-10 as an anti-inflammatory drug: Several studies have tested the usefulness of IL-10 as an anti-inflammatory agent to reduce the risk of inflammation associated PTB.^{2,45} Deficiency of IL-10 in the amniotic fluid of women with intraamniotic infection has been reported as a major factor associated with fetal inflammation and PTB. In vitro models^{2,46-52} and in vivo non-human primate animal models and mouse models⁵³⁻⁵⁵. Unfortunately, the requirement of intraamniotic administration of IL-10 to reduce fetal inflammation has hindered its advancement in clinical trials.

Extracellular vesicles as drug delivery vehicles: Recently, the inventors have developed a novel technology to engineer extracellular vesicles (EVs-exosomes of 40-175 nm) using electroporation approaches that can cross placenta, reduce fetal inflammatory response. Using this technology, the inventors have shown that anti-inflammatory NF-kB inhibitor drug encoded in EVs can cross placental barriers to reduce PTB. This anti-inflammatory NF-kB inhibitor drug was shown to result in delaying PTB in infection induced PTB model in mice.

The present invention uses an effective intervention to a deliver specific and natural anti-inflammatory agent, IL-10. The inventors developed an IL-10 engineered in EVs.

Currently, no effective treatment exists for infection/inflammation-associated PTB where fetal inflammatory response is one of the major effectors. Understanding fetal-specific innate cell-associated inflammatory response and its reduction is vital. Drug delivery techniques using naturally occurring, inert, nanoparticles that are immunologically inert will be vital in addressing PTB syndrome.

It was found that the engineered exosomes are a safe, stable, and specific intervention to minimize the risk of inflammation-associated PTB. Successful testing of this drug will help to develop an investigational new drug status and use UTMB's Maternal-Fetal Medicine Unit Network Center for future clinical trials. Engineer EVs can contain desired proteins and test its usefulness as drug delivery vehicles. EVs are inert and nonimmunogenic and make its usefulness ideal for conditions like PTB where placenta acts as barrier for most of the currently available drugs.

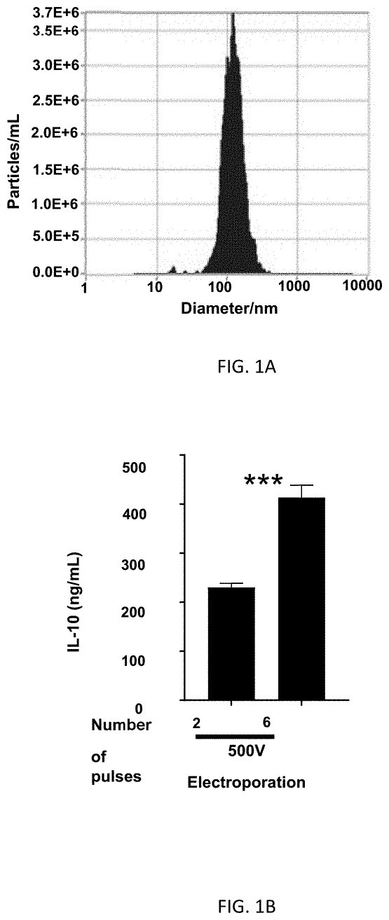

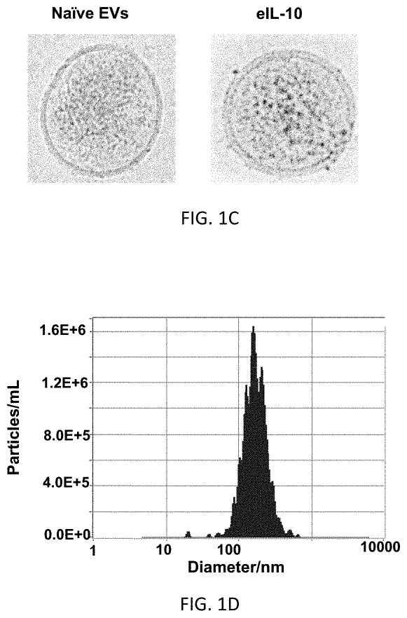

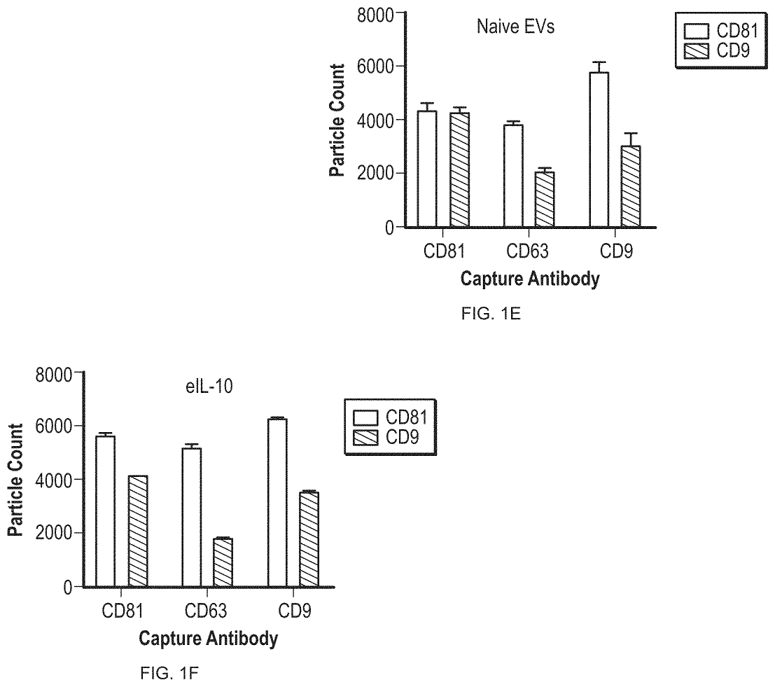

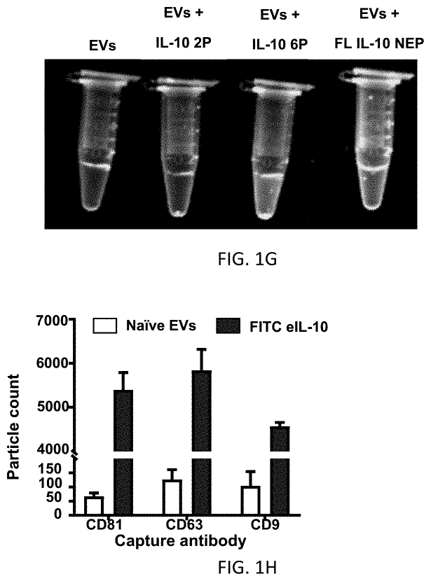

EVs or exosomes are EVs with a size range between 40 nm-175 nm. The inventors have successfully made exosomes that contain IL-10 in the exosomal lumen. Using amnion epithelial cell derived exosomes⁵⁶⁻⁵⁹, the inventors conducted multiple electroporation experiments to load recombinant IL-10 into the exosomal lumen. A step-by-step approach of IL-10 in exosome or “drug” developmental strategies and testing of functional IL-10 are shown in FIGS. 1A to 1H.

FIGS. 1A to 1H: Properties of naïve EVs vs EVs encapsulating IL-10. FIG. 1A. Nanoparticle Tracking Analysis using ZetaView showing EV size in nanometers prior to electroporation. FIG. 1B. Optimization of electroporation protocol. IL-10 was encapsulated in EVs via sonication or electroporation at 500 Volts with either 2 or 6 pulses. Six pulses at 500 V were optimum and provided the highest loading of IL-10 within EVs. These conditions were used for the generation of eIL10 in further experiments. FIG. 1C. Cryo EM images showing the morphology of EVs. Representative images of naïve and electroporated EVs show similar morphology. Both EVs were round with double layered membranes. FIG. 1D. Nanoparticle Tracking Analysis using ZetaView showing EV size in nanometers after electroporation. The particles do not show a significant change in size after electroporation compared to nonelectroporated EVs (1A). FIG. 1E. ExoView analysis of EV markers: CD81 (red), CD63, and CD9 (green) were determined by ExoView and confirmed the presence of EV markers that are not altered by electroporation or encapsulation of IL-10. FIG. 1F. Confirmation of IL-10 loading in EVs. Recombinant (r) IL-10 was conjugated with FITC and its presence in EVs was determined by fluorescence under UV light. Naïve EVs and EVs that were electroporated with only 2 pulses do not show fluorescence. EVs electroporated with 6 pulses, or EVs mixed with IL-10 without electroporation show higher levels of fluorescence. FIG. 1G. Confirmation of IL-10 loading in EVs using Exoview analysis. EVs captured to ExoView chips using tetraspanin markers were probed with IL-10 antibody to detect its luminal presence. FIG. 1H. Compared to naïve exosomes (white), eIL-10 EVs (blue) show presence of IL-10, and loading efficacy was calculated up to 70%. Data are shown as means±SEM. For all groups, n≥5. Data are shown as means±SEM. Data were analyzed using a one-way ANOVA with a Tukey's post hoc test. ***P≤0.001.

Electroporation technology was used to engineer exosomes to contain IL-10. IL-10 was actively incorporated into exosomes via (eIL-10) electroporation. Data was generated to test the efficacy of the eIL-10.

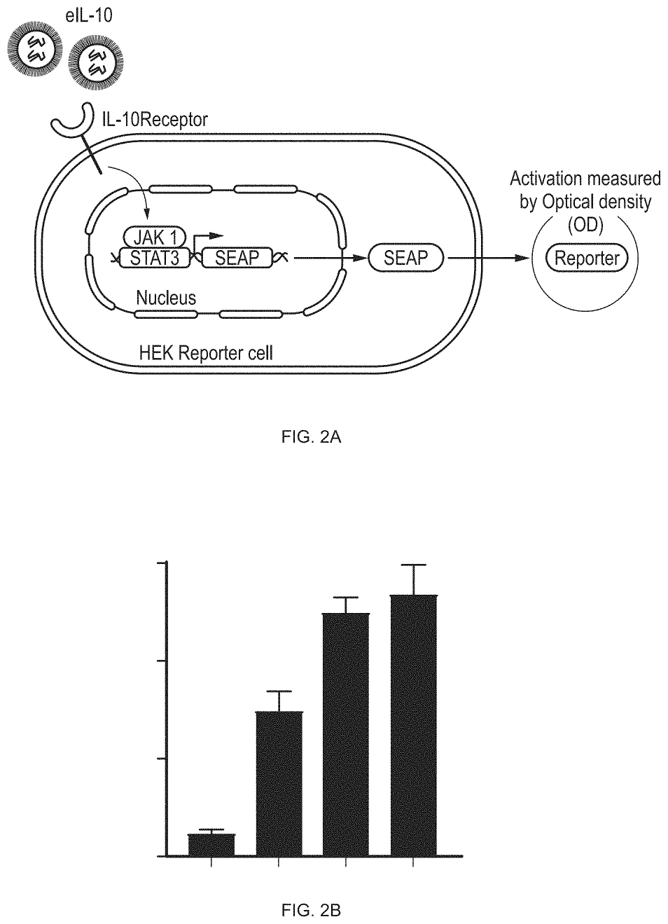

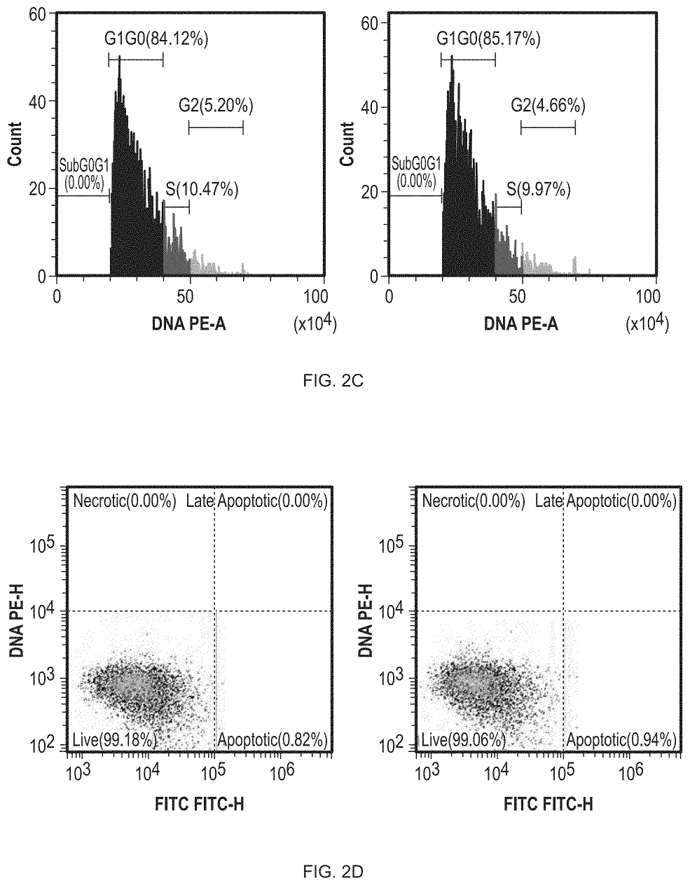

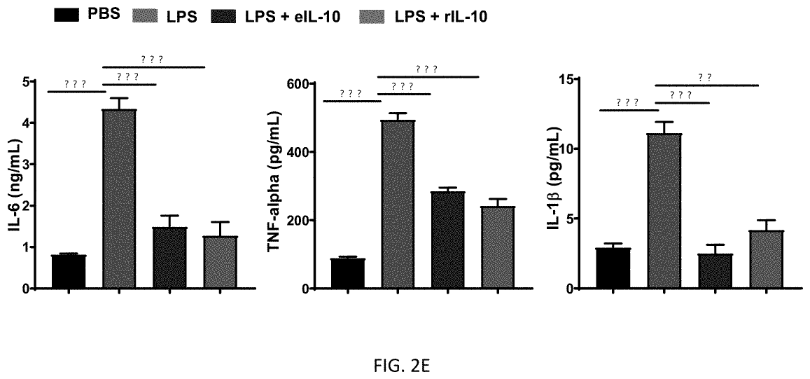

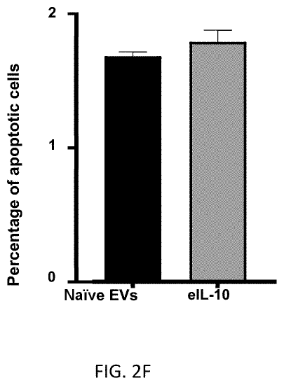

FIGS. 2A to 2F: Activity of eIL10 at IL-10 pathway and cytokine production. FIG. 2A. Schematic of secreted embryonic alkaline phosphatase (SEAP) reporter assay to determine the functional properties of IL-10 in EVs. HEK cells transfected with SEAP reporter where IL-10/IL-10 receptor binding leads to signaling activation and causing a color change. The color change can be measured by optical density. FIG. 2B. Optical density (OD) measured in SEAP-transfected cells. Naïve EVs does not contain IL-10. eIL10 shows binding to the receptor in a dose-dependent manner, contributing to SEAP activation as detected by increased OD. FIG. 2C. Determining the impact of eIL-10 on cell cycle using flow cytometry. Cell cycle analysis was performed in RAW264.7 (mouse macrophage) cells treated with naïve EVs or eIL10. Flow cytometry showed no significant changes in the number of cells found in each stage of the cell cycle. FIG. 2D. Determining the impact of eIL-10 on cell fate using flow cytometry. Analysis of apoptosis and necrosis using Annexin and propidium iodide staining in RAW cells treated with naïve EVs or eIL10. Neither naïve nor eIL-10 produced any necrosis of cells. Similarly, apoptotic cell deaths were <1% in both naïve and eIL-10 treated cells. FIG. 2E. Functional properties of eIL-10. ELISA was used to measure the levels of inflammatory cytokines produced by RAW264.7 cells (Mouse macrophages) treated with LPS and co-treated with either eIL10 or rIL10. LPS (red)-induced IL-6, TNF-a, and IL-1b co-treatment with eIL-10 (blue) rIL-10 (green) reduced the production of pro-inflammatory cytokines confirming functionally viable IL-10 inside the EVs. For all groups, n≥5. FIG. 2F. Percentage of apoptotic cells naïve EVs v eIL-10. Data are shown as means±SEM. Data were analyzed using a one-way ANOVA with a Tukey's post hoc test. *P≤0.05, **P≤0.01, ***P≤0.001, ****P≤0.0001.

Methods to load IL-10 in exosomes (eIL-10). Exosomes in this study were extracellular vesicles with a size range between <40 nm-160 nm from HEK293T cells. HEK293T cells were cultured, exosomes were isolated from media by ultracentrifugation, and purification and size exclusion chromatography were performed to obtain exosomes of defined range. Exosomes were quantitated using a Zeta view analyser, and particle sizes ranging between 40 nm-110 nm were isolated for further use. To confirm the efficiency of loading and particle size, the inventors used ExoView® (NanoView Bioscience, CA), which can distinguish between EV subpopulations defined by size and protein marker expression. ExoView (NanoView Bioscience, CA) data suggest that the eIL-10 of the present invention had a mean particle size of 80 nm, and efficiency of IL-10 loading was ˜73%. The results demonstrate that eIL-10 are stable and functionally viable at −80° C. for up to a year.

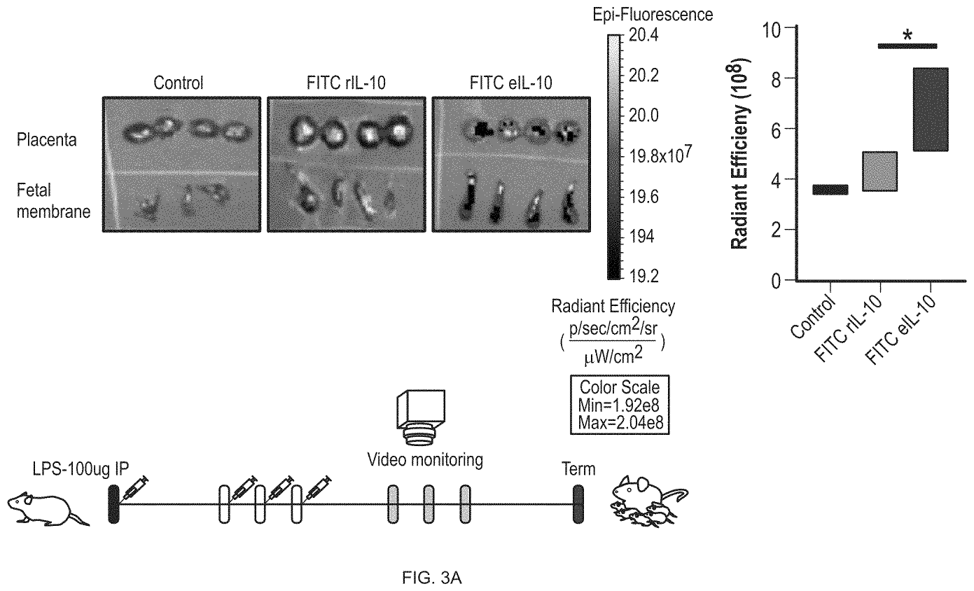

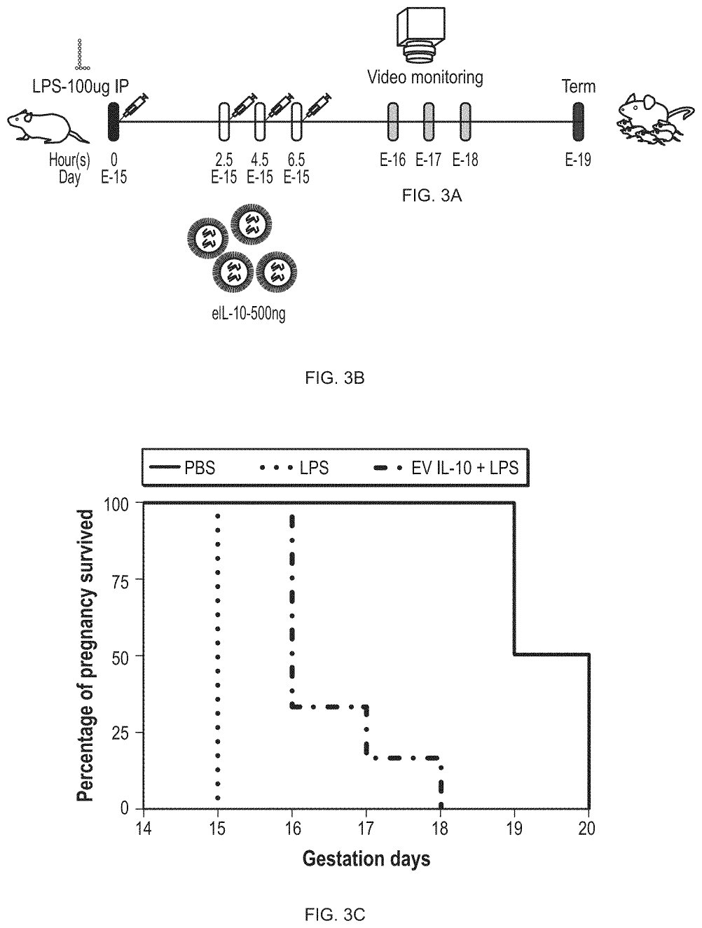

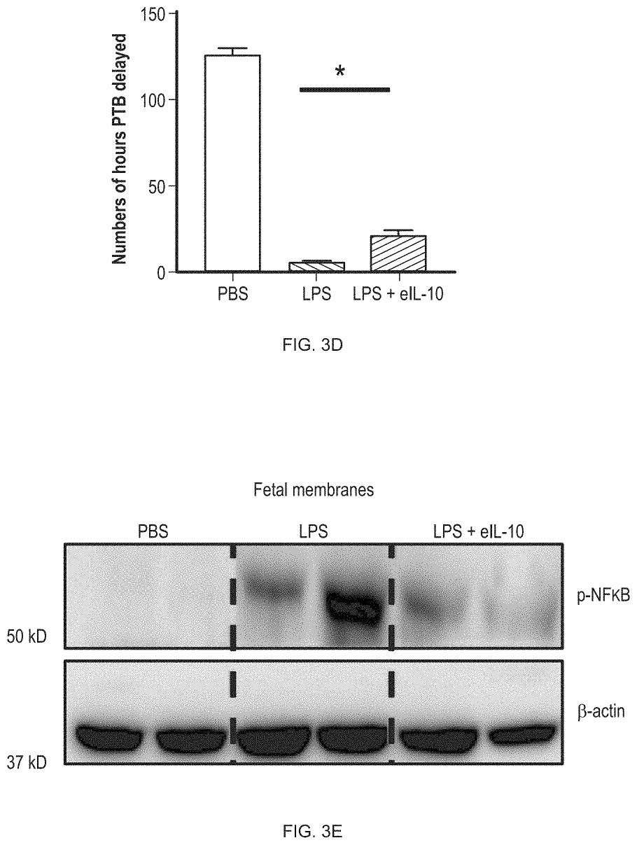

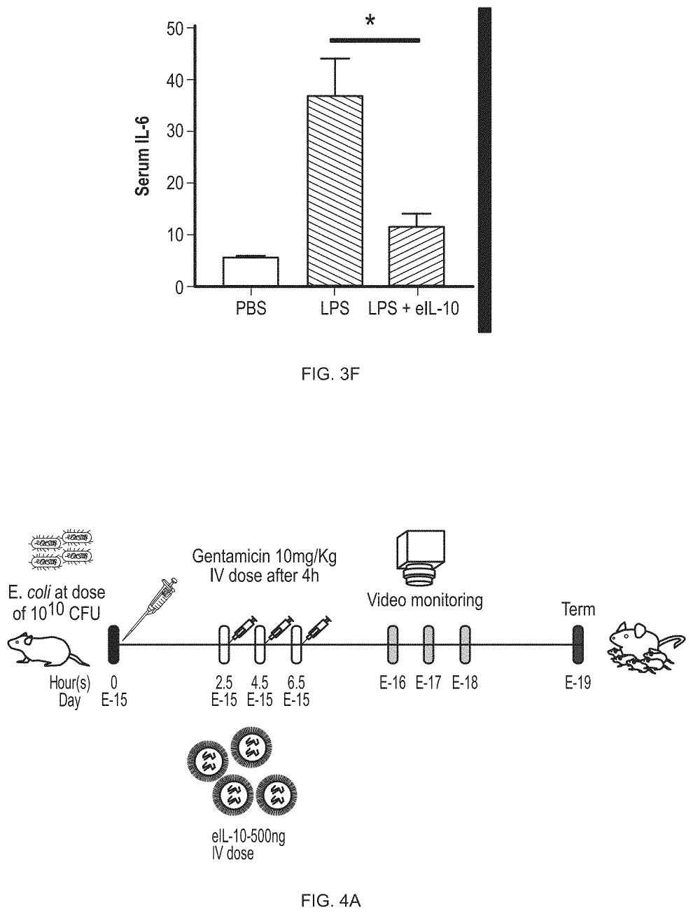

FIGS. 3A to 3F: Bioavailability and effect of eIL10 in LPS-induced PTB mouse model. FIG. 3A. Left panel: Representative In vivo imaging system (IVIS) images. Fluorescently labeled IL-10 is seen in the placenta and fetal membrane when it is packaged in the EVs (FITC eIL-10) but not when rIL-10 was injected directly (FITC rIL-10). Controls remain negative, Right panel: Radiant efficiency in target tissues is shown for eIL10 (red) and rIL-10 (green). FIG. 3B. Schematic representation of LPS- (100 μg) (intraperitoneally injected) induced PTB model and its treatment using eIL-10 (500 ng) (intravenously injected through the tail vain). FIG. 3C. Survival curve comparing PBS (black), LPS (red) and LPS+eIL-10 (blue) treatment groups. LPS injections caused PTB within 24 hours, while LPS+eIL-10 delayed PTB by an average of 24 hours, compared to controls. For all groups, n≥8. FIG. 3D. Bar graph of number of hours of pregnancy maintained after eIL-10 treatment. eIL-10 delays PTB in LPS-challenged pregnant CD-1 mice. FIG. 3E. Representative western blot images of fetal membranes collected 6 hours after treatment with LPS and eIL-10. eIL-10 treatment reduced NF-κB activation (reduction of Phosphorylated (p) NF-κB) compared to LPS. FIG. 3F. ELISA of maternal plasma from blood collected 6 hours after treatment shows a decrease in IL-6 in mice treated with eIL10 compared to LPS. For all groups, n≥5. Data are shown as means±SEM. Data were analyzed using a one-way ANOVA with a Tukey's post hoc test. *P≤0.05, **P≤0.01, ***P≤0.001, ****P≤0.0001.

Characterization, optimization, and determining eIL-10 efficacy. Two separate studies were performed to characterize and optimize the conditions for efficient loading of IL-10 in exosomes. The concentration of IL-10 in exosomes was determined by ELISA. eIL-10 was characterized by Exoview for IL-10 loading efficiency using exosomal protein markers as proxies. Additionally, data from an in vitro model was determined and co-treatment of eIL-10 in both fetal and maternal (decidua) cells (amnion mesenchymal cells) show a reduction of LPS induced cytokines TNF-alpha and IL-6.

Intraamniotic injection of eIL-10 delays LPS-induced PTB. On E-15, pregnant CD-1 mice (n=5 in each group) were injected intraamniotically with one of the following: PBS, LPS (100 μg/100 μL), LPS+recombinant IL-10 (200 ng) or LPS+eIL-10 (200 ng) in each amniotic sac of uterus. LPS induced PTB in these animals within 8 hours compared to PBS-injected animals (term delivery). Animals injected with eIL-10 delayed PTB -24 hours compared with LPS. This is equivalent to almost 10 days in human pregnancy. A second batch of animals was sacrificed 6 hours after LPS or eIL-10+LPS administration, and plasma and tissues were collected. LPS-induced NF-κB activation was significantly decreased in the fetal membranes of mice injected with eIL-10+LPS compared to LPS-injected mice as indicated by decreased NF-κB activation (P-Rel A subunit). Additionally, maternal plasma IL-6 levels were significantly decreased in eIL-10+LPS injected mice. These results were consistent with Novy and Gravett's group, who showed that intraamniotic injection of IL-10 minimizes the incidence of inflammation-induced PTB in rhesus monkeys. Since eIL-10 can cross placental barriers and reach fetal tissues, experiments can be conducted using IV and IM routes to overcome one of the limitations of administering the drug intraamniotically, which have been shows by the present inventors to show efficacy of an NF-kB inhibitor to cross the F-M membrane.

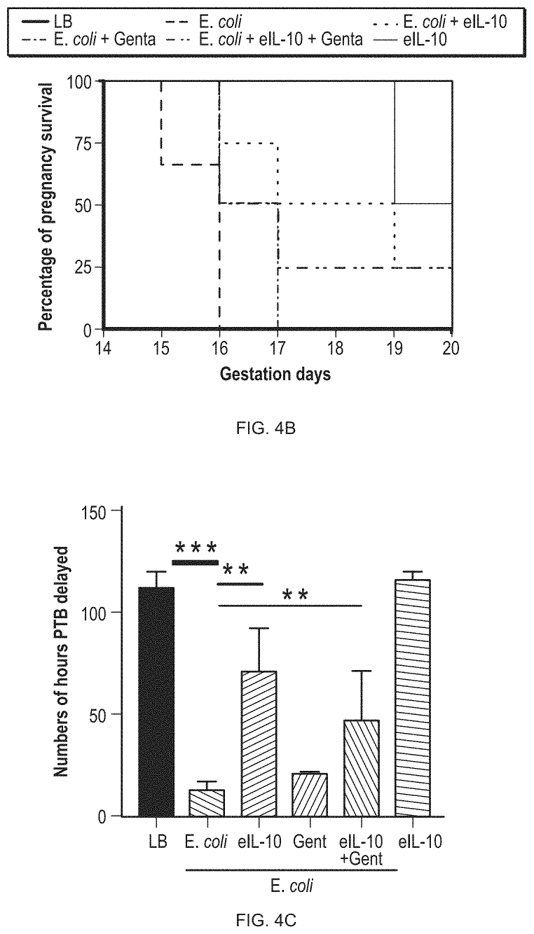

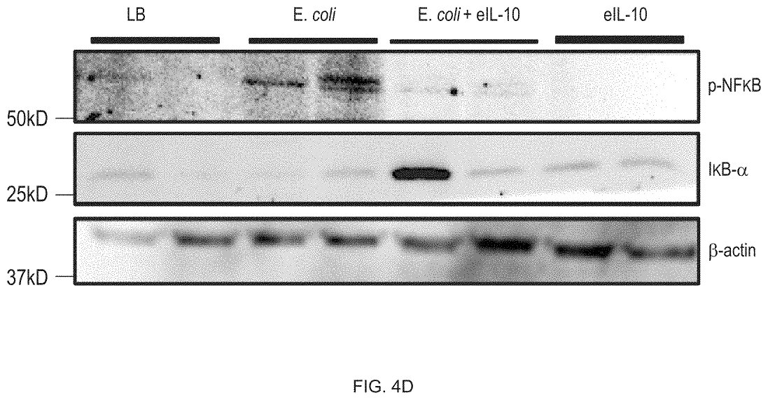

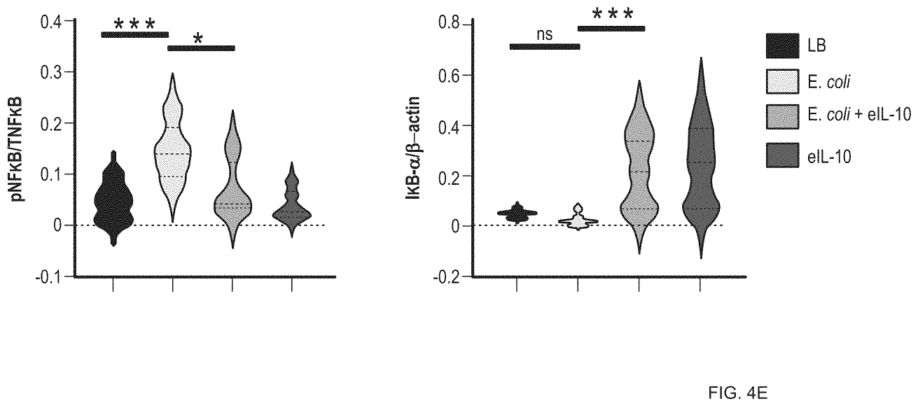

FIGS. 4A to 4E: Effect of eIL10 in E. coli-induced ascending infection mouse model. FIG. 4A. Schematic representation of ascending infection-induced preterm birth mouse model and intravenous treatment with eIL10 (500 ng) and/or gentamicin (10 mg/kg). Infection is induced by vaginal administration of E. coli (1010 colony forming units [CFU]). FIG. 4B. Survival graph comparing liquid broth, LB (black), E. coli (red), E. coli+eIL-10 (blue), E. coli+gentamicin (green), E. coli+eIL-10+gentamicin (purple) and only eIL-10 (pink) treatment groups. E. coli treatment caused PTB within 48 hours, while E. coli+eIL-10 delayed PTB to term delivery with live pups whereas gentamicin treatment delays only 24 hours compared to E. coli group. For all groups, n≥4. FIG. 4C. Delay in preterm birth (in hours) shown for each treatment group. Color key as seen in FIG. 4B. A significant delay in preterm birth is shown in mice treated with eIL10 or eIL10 and gentamicin compared to challenge with E. coli. FIG. 4D. Western blot of fetal membranes collected from pregnant mice 24 hours after treatment. Pregnant mice were treated with vehicle liquid broth (LB), E. coli, co-treatment of E. coli and eIL10, or eIL10 without E. coli challenge. E. coli-induced increase of activated NF-κB (phosphorylated: pNF-κB) is reversed with eIL10 administration. I-κB-α (an inhibitor of NF-κB is increased with treatment of eIL10. FIG. 4E. Western blot of fetal membranes showing levels of phosphorylated NF-κB normalized to total NF-κB and I-κB-α normalized to β-actin in fetal membranes collected from mice 24 hours after treatment as measured in western blot. Color key as seen in FIG. 4B. Immune challenge tests: Serum levels of IL-6 production measured by ELISA in pups born after exposure to E. coli, eIL10, and gentamicin treatment in utero. No significant changes are seen in IL-6 production in pups regardless of exposure indicative of lack of long term immune supression. Levels of TNF-α production in pups exposed to E. coli, eIL10, and gentamicin treatment in utero measured by ELISA. Color key as seen in FIG. 4B. No significant changes are seen in pups' TNF-α production. For all groups, n≥5. Data are shown as means±SEM. Data were analyzed using a one-way ANOVA with a Tukey's post hoc test.

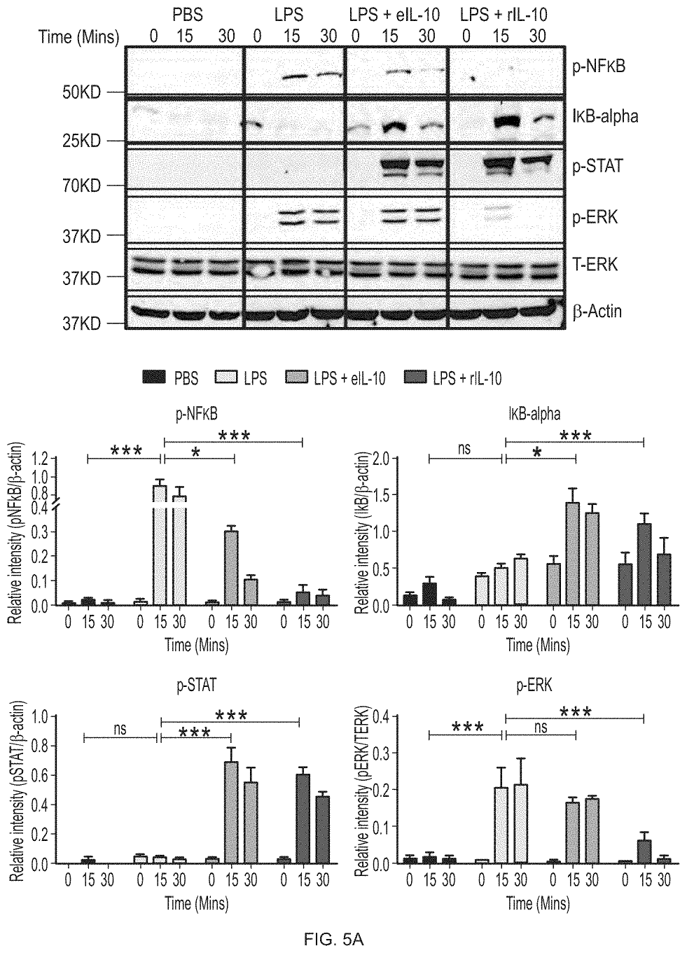

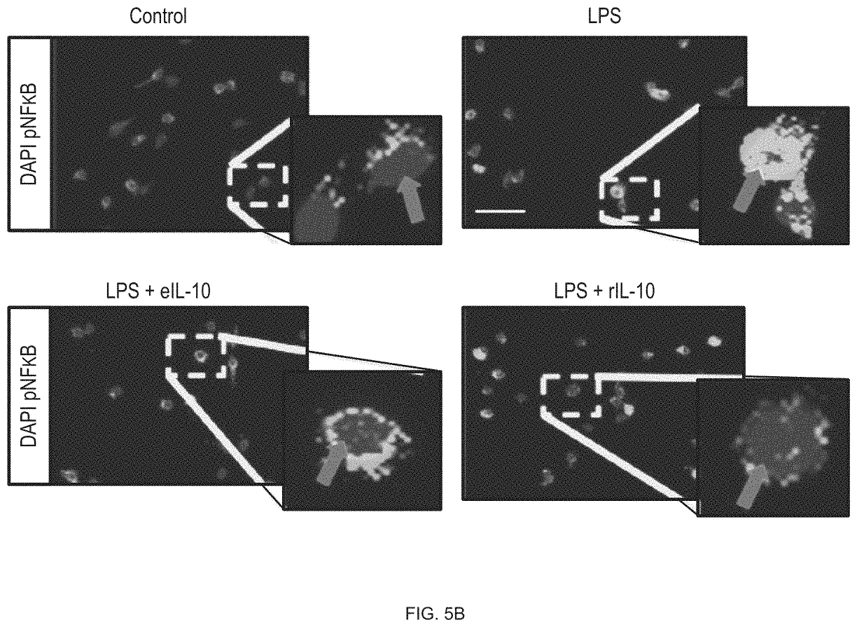

FIGS. 5A and 5B: eIL-10 inhibits NF-κB pathway. FIG. 5A. Representative western blot images of cell lysates collected from RAW264.7 (mouse macrophage) cells treated with LPS (red bar) and co-treated with either eIL10 (blue bar) or rIL10 (green bar) for a treatment time of 0, 15, or 30 minutes. LPS-induced increases of activated NF-κB (phosphorylated: p-NF-κB) and activated ERK (phosphorylated: p-ERK) were reversed by treatment with eIL10 or rIL10. Co-treatment of eIL10 or rIL10 also increased levels of IκB-α (inhibitor of NF-κB) and phosphorylated STAT (p-STAT). Bar graphs show relative intensity of these bands compared to β-actin, and phosphorylated ERK shows relative intensity against total ERK. All the experiments were repeated three times. Normalized data were shown as means±SEM. Data were analyzed using a one-way ANOVA with a Tukey's post hoc test. *P≤0.05, **P≤0.01, ***P≤0.001, ****P≤0.0001. FIG. 5B. Immunocytochemistry staining of RAW264.7 cells with activated NF-κB (phosphorylated: pNF-κB) antibody (green) and DAPI (blue) to show translocation of p-NF-κB into the nucleus.

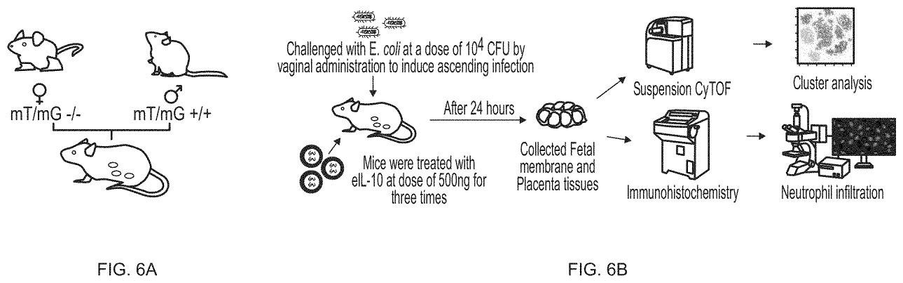

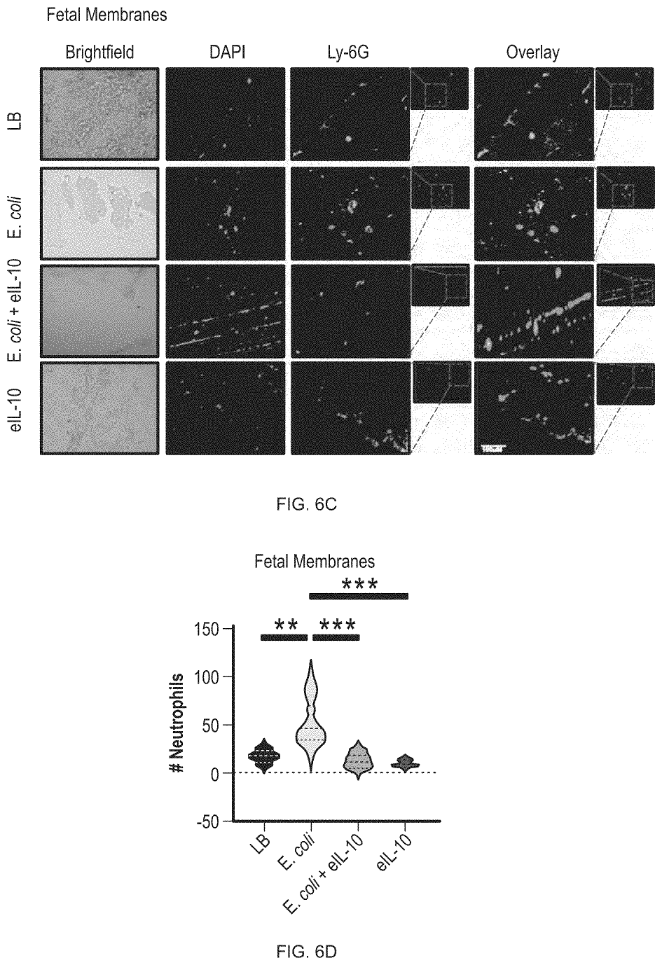

FIGS. 6A to 6D: eIL10 treatment reduced neutrophil infiltration and HCA. FIG. 6A. Schematic representation of a transgenic animal model with a membrane-targeted, 2-color fluorescent Cre-reporter allele where membrane-expressed tandem dimer Tomato (tdTomato—mT+). mT+ males were mated with wild-type female (WT) mice, all fetal tissues had the mT/mG construct expressing mT+ (red fluorescence), keeping maternal tissues negative. FIG. 6B. Schematic representation of E. coli challenge and treatment of mice, various approaches of analysis of samples and data collection. Using the pregnant mouse model (FIG. 6A), mice are treated with vaginal administration of 104 colony forming units (CFU) of E. coli to induce ascending infection. Co-treatment with eIL10 is administered at a dose of 500 ng (intravenous through tail vein). FIG. 6C. Immunohistochemistry of fetal membranes 24 hours after administration of vehicle liquid broth (LB), E. coli challenge, co-treatment of E. coli with eIL10, or eIL10 without E. coli challenge. Tissues were stained with Ly-6G (green) for neutrophils and DAPI (blue) to show nuclei. FIG. 6D. Violin graphs show significant reduction in neutrophil levels by eIL10 (blue) after challenge with E. coli (red) as seen in IHC fluorescent microscopy. The number of neutrophils in fetal membranes is reduced to levels similar to those seen in vehicle liquid broth (LB, black) and eIL10 treatment alone (pink). For all groups, n≥5. Data are shown as means±SEM. Data were analyzed using a one-way ANOVA with a Tukey's post hoc test. *P≤0.05, **P≤0.01, ***P≤0.001, ****P≤0.0001.

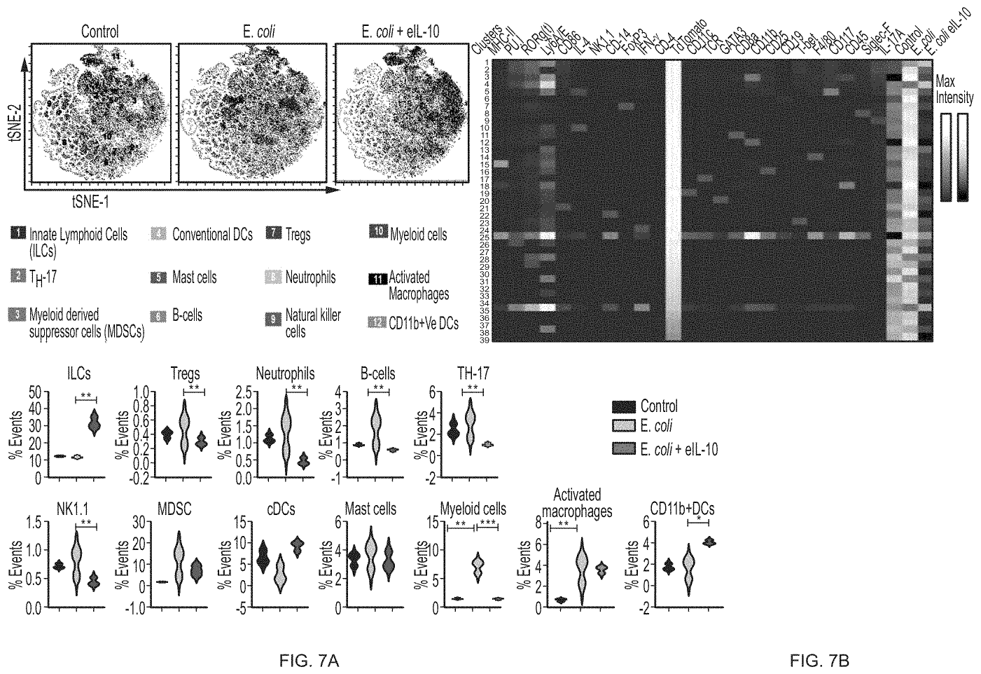

FIGS. 7A and 7B. Fetal specific Immune cell characterization during pregnancy in different treatment groups after 24 hours by mass cytometer. FIG. 7A. Identify differentially distributed cellular phenotypes by t-SNE in concatenated control, E. coli and E. coli+eIL-10 treated groups, respectively. FIG. 7B. The heatmaps shows the relative intensity of each parameter for different clusters in different groups. The percentage events of the particular cluster were determined by the bar charts from the cluster explorer tool and cluster was characterized by the expression profile expression markers. The characterized clusters percentage events were compared between different treatment groups. For all groups, n≥5. Data are shown as means±SEM. Data were analyzed using a one-way ANOVA with a Tukey's post hoc test. *P≤0.05, **P≤0.01, ***P≤0.001, ****P≤0.0001.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains.

All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Beck S, Wojdyla D, Say L, et al. The worldwide incidence of preterm birth: a systematic review of maternal mortality and morbidity. Bull World Health Organ. 2010; 88(1): 31-38.

2. Dudley D J. Pre-term labor: an intra-uterine inflammatory response syndrome? J Reprod Immunol. 1997; 36(1-2): 93-109.

3. Chaiworapongsa T, Romero R, Kim J C, et al. Evidence for fetal involvement in the pathologic process of clinical chorioamnionitis. Am J Obstet Gynecol. 2002; 186(6): 1178-1182.

4. Romero R, Savasan Z A, Chaiworapongsa T, et al. Hematologic profile of the fetus with systemic inflammatory response syndrome. J Perinat Med. 2011; 40(1): 19-32.

5. Goncalves L F, Chaiworapongsa T, Romero R. Intrauterine infection and prematurity. Ment Retard Dev Disabil Res Rev. 2002; 8(1): 3-13.

6. Yoon B H, Romero R, Park J S, et al. The relationship among inflammatory lesions of the umbilical cord (funisitis), umbilical cord plasma interleukin 6 concentration, amniotic fluid infection, and neonatal sepsis. Am J Obstet Gynecol. 2000; 183(5): 1124-1129.

7. Svensson-Arvelund J, Mehta R B, Lindau R, et al. The human fetal placenta promotes tolerance against the semiallogeneic fetus by inducing regulatory T cells and homeostatic M2 macrophages. J Immunol. 2015; 194(4): 1534-1544.

8. Schumacher A, Sharkey D J, Robertson S A, Zenclussen A C. Immune Cells at the Fetomaternal Interface: How the Microenvironment Modulates Immune Cells To Foster Fetal Development. J Immunol. 2018; 201(2): 325-334.

9. Negishi Y, Takahashi H, Kuwabara Y, Takeshita T. Innate immune cells in reproduction. J Obstet Gynaecol Res. 2018; 44(11): 2025-2036.

10. Alijotas-Reig J, Llurba E, Gris J M. Potentiating maternal immune tolerance in pregnancy: a new challenging role for regulatory T cells. Placenta. 2014; 35(4): 241-248.

11. Chavan A R, Griffith O W, Wagner G P. The inflammation paradox in the evolution of mammalian pregnancy: turning a foe into a friend. Curr Opin Genet Dev. 2017; 47: 24-32.

12. Mor G, Cardenas I, Abrahams V, Guller S. Inflammation and pregnancy: the role of the immune system at the implantation site. Ann N Y Acad Sci. 2011; 1221: 80-87.

13. Kshirsagar S K, Alam S M, Jasti S, et al. Immunomodulatory molecules are released from the first trimester and term placenta via exosomes. Placenta. 2012; 33(12): 982-990.

14. Gomez-Lopez N, StLouis D, Lehr M A, Sanchez-Rodriguez E N, Arenas-Hernandez M. Immune cells in term and preterm labor. Cell Mol Immunol. 2014; 11(6): 571-581.

15. Lash G E. Molecular Cross-Talk at the Feto-Maternal Interface. Cold Spring Harb Perspect Med. 2015; 5(12).

16. Liu S, Diao L, Huang C, Li Y, Zeng Y, Kwak-Kim J Y H. The role of decidual immune cells on human pregnancy. J Reprod Immunol. 2017; 124: 44-53.

17. Schliefsteiner C, Peinhaupt M, Kopp S, et al. Human Placental Hofbauer Cells Maintain an Anti-inflammatory M2 Phenotype despite the Presence of Gestational Diabetes Mellitus. Front Immunol. 2017; 8: 888.

18. Erlebacher A. Immunology of the maternal-fetal interface. Annu Rev Immunol. 2013; 31: 387-411.

19. Nancy P, Erlebacher A. [Epigenetic repression of chemokine expression at the maternal-fetal interface as a mechanism of feto-maternal tolerance]. Med Sci (Paris). 2012; 28(12): 1037-1039.

20. Goldenberg R L, Culhane J F, Jams J D, Romero R. Epidemiology and causes of preterm birth. Lancet. 2008; 371(9606): 75-84.

21. Dudley D J. Immunoendocrinology of preterm labor: the link between corticotropin-releasing hormone and inflammation. Am J Obstet Gynecol. 1999; 180(1 Pt 3): 5251-256.

22. Romero R, Dey S K, Fisher S J. Preterm labor: one syndrome, many causes. Science. 2014; 345(6198): 760-765.

23. Gomez-Lopez N, Vadillo-Perez L, Hernandez-Carbajal A, Godines-Enriquez M, Olson D M, Vadillo-Ortega F. Specific inflammatory microenvironments in the zones of the fetal membranes at term delivery. Am J Obstet Gynecol. 2011; 205(3): 235-224.

24. Romero R, Gotsch F, Pineles B, Kusanovic J P. Inflammation in pregnancy: its roles in reproductive physiology, obstetrical complications, and fetal injury. Nutr Rev. 2007; 65(12 Pt 2): S194-S202.

25. Bukowski R, Sadovsky Y, Goodarzi H, et al. Onset of human preterm and term birth is related to unique inflammatory transcriptome profiles at the maternal fetal interface. PeerJ. 2017; 5: e3685.

26. Rinaldi S F, Makieva S, Saunders P T, Rossi A G, Norman J E. Immune cell and transcriptomic analysis of the human decidua in term and preterm parturition. Mol Hum Reprod. 2017; 23(10): 708-724.

27. Xu Y, Romero R, Miller D, et al. Innate lymphoid cells at the human maternal-fetal interface in spontaneous preterm labor. Am J Reprod Immunol. 2018; 79(6): e12820.

28. Hamilton S, Oomomian Y, Stephen G, et al. Macrophages infiltrate the human and rat decidua during term and preterm labor: evidence that decidual inflammation precedes labor. Biol Reprod. 2012; 86(2): 39.

29. Peltier M R. Immunology of term and preterm labor. Reprod Biol Endocrinol. 2003; 1: 122.

30. Zhang J, Shynlova O, Sabra S, Bang A, Briollais L, Lye S J. Immunophenotyping and activation status of maternal peripheral blood leukocytes during pregnancy and labour, both term and preterm. J Cell Mol Med. 2017; 21(10): 2386-2402.

31. Edey L F, O'Dea K P, Herbert B R, et al. The Local and Systemic Immune Response to Intrauterine LPS in the Prepartum Mouse. Biol Reprod. 2016; 95(6): 125.

32. Osman I, Young A, Jordan F, Greer I A, Norman J E. Leukocyte density and proinflammatory mediator expression in regional human fetal membranes and decidua before and during labor at term. J Soc Gynecol Investig. 2006; 13(2): 97-103.

33. Cappelletti M, Presicce P, Lawson M J, et al. Type I interferons regulate susceptibility to inflammation-induced preterm birth. JCI Insight. 2017; 2(5): e91288.

34. Boonkasidecha S, Kannan P S, Kallapur S G, Jobe A H, Kemp M W. Fetal skin as a pro-inflammatory organ: Evidence from a primate model of chorioamnionitis. PLoS One. 2017; 12(9): e0184938.

35. Hinz M, Scheidereit C. The IkappaB kinase complex in NF-kappaB regulation and beyond. EMBO Rep. 2014; 15(1): 46-61.

36. Chen J, Chen Z J. Regulation of NF-kappaB by ubiquitination. Curr Opin Immunol. 2013; 25(1): 4-12.

37. Keelan J A, Blumenstein M, Helliwell R J, Sato T A, Marvin K W, Mitchell M D. Cytokines, prostaglandins and parturition—a review. Placenta. 2003; 24 Suppl A: S33-46.

38. Mendelson C R. Minireview: fetal-maternal hormonal signaling in pregnancy and labor. Mol Endocrinol. 2009; 23(7): 947-954.

39. Shynlova O, Tsui P, Jaffer S, Lye S J. Integration of endocrine and mechanical signals in the regulation of myometrial functions during pregnancy and labour. Eur J Obstet Gynecol Reprod Biol. 2009; 144 Suppl 1: S2-10.

40. Golightly E, Jabbour H N, Norman J E. Endocrine immune interactions in human parturition. Mol Cell Endocrinol. 2011; 335(1): 52-59.

41. Trivedi S, Joachim M, McElrath T, et al. Fetal-placental inflammation, but not adrenal activation, is associated with extreme preterm delivery. Am J Obstet Gynecol. 2012; 206(3): 236 e231-238.

42. Iliodromiti Z, Antonakopoulos N, Sifakis S, et al. Endocrine, paracrine, and autocrine placental mediators in labor. Hormones (Athens). 2012; 11(4): 397-409.

43. Buhimschi C S, Turan O M, Funai E F, et al. Fetal adrenal gland volume and cortisol/dehydroepiandrosterone sulfate ratio in inflammation-associated preterm birth. Obstet Gynecol. 2008; 111(3): 715-722.

44. Romero R, Espinoza J, Kusanovic J P, et al. The preterm parturition syndrome. BJOG. 2006; 113 Suppl 3: 17-42.

45. Gotsch F, Romero R, Kusanovic J P, et al. The anti-inflammatory limb of the immune response in preterm labor, intra-amniotic infection/inflammation, and spontaneous parturition at term: a role for interleukin-10. J Matern Fetal Neonatal Med. 2008; 21(8): 529-547.

46. Zaga-Clavellina V, Garcia-Lopez G, Flores-Herrera H, et al. In vitro secretion profiles of interleukin (IL)-1beta, IL-6, IL-8, IL-10, and TNF alpha after selective infection with Escherichia coli in human fetal membranes. Reprod Biol Endocrinol. 2007; 5: 46.

47. Zaga-Clavellina V, Flores-Espinosa P, Pineda-Torres M, et al. Tissue-specific IL-10 secretion profile from term human fetal membranes stimulated with pathogenic microorganisms associated with preterm labor in a two-compartment tissue culture system. J Matern Fetal Neonatal Med. 2014; 27(13): 1320-1327.

48. Zaga-Clavellina V, Garcia-Lopez G, Flores-Herrera H, et al. In vitro secretion profiles of interleukin (IL)-1beta, IL-6, IL-8, IL-10, and TNF alpha after selective infection with Escherichia coli in human fetal membranes. Reprod Biol Endocrinol. 2007; 5: 46.

49. Fortunato S J, Menon R, Lombardi S J. Interleukin-10 and transforming growth factor-beta inhibit amniochorion tumor necrosis factor-alpha production by contrasting mechanisms of action: therapeutic implications in prematurity. Am J Obstet Gynecol. 1997; 177(4): 803-809.

50. Fortunato S J, Menon R, Lombardi S J. The effect of transforming growth factor and interleukin-10 on interleukin-8 release by human amniochorion may regulate histologic chorioamnionitis. Am J Obstet Gynecol. 1998; 179(3 Pt 1): 794-799.

51. Fortunato S J, Menon R, Lombardi S J, LaFleur B. Interleukin-10 inhibition of gelatinases in fetal membranes: therapeutic implications in preterm premature rupture of membranes. Obstet Gynecol. 2001; 98(2): 284-288.

52. Fortunato S J, Menon R, Swan K F, Lombardi S J. Interleukin-10 inhibition of interleukin-6 in human amniochorionic membrane: transcriptional regulation. Am J Obstet Gynecol. 1996; 175(4 Pt 1): 1057-1065.

53. Robertson S A, Care A S, Skinner R J. Interleukin 10 regulates inflammatory cytokine synthesis to protect against lipopolysaccharide-induced abortion and fetal growth restriction in mice. Biol Reprod. 2007; 76(5): 738-748.

54. Robertson S A, Skinner R J, Care A S. Essential role for IL-10 in resistance to lipopolysaccharide-induced preterm labor in mice. J Immunol. 2006; 177(7): 4888-4896.

55. Sadowsky D W, Novy M J, Witkin S S, Gravett M G. Dexamethasone or interleukin-10 blocks interleukin-1beta-induced uterine contractions in pregnant rhesus monkeys. Am J Obstet Gynecol. 2003; 188(1): 252-263.

56. Sheller-Miller S, Choi K, Choi C, Menon R. Cyclic-recombinase-reporter mouse model to determine exosome communication and function during pregnancy. Am J Obstet Gynecol. 2019; 221(5): 502 e501-502 e512.

57. Sheller-Miller S, Lei J, Saade G, Salomon C, Burd I, Menon R. Feto-Maternal Trafficking of Exosomes in Murine Pregnancy Models. Frontiers in Pharmacology. 2016; 7.

58. Sheller-Miller S, Radnaa E, Arita Y, et al. Environmental pollutant induced cellular injury is reflected in exosomes from placental explants. Placenta. 2020; 89: 42-49.

59. Sheller-Miller S, Trivedi J, Yellon S M, Menon R. Exosomes Cause Preterm Birth in Mice: Evidence for Paracrine Signaling in Pregnancy. Sci Rep. 2019; 9(1): 608.

What is claimed is:

1. A method of treating a pregnant female subject at risk of preterm birth, comprising administering to the pregnant female subject an effective amount of an exosome that comprises a nucleic acid that expresses IL-10 or that comprises IL-10, wherein the effective amount of the IL-10 is effective to reduce, prevent or delay preterm birth.

2. The method of claim 1, wherein the preterm birth is spontaneous preterm birth.

3. The method of claim 1, wherein the pregnant human female presents to labor and delivery with symptoms of preterm birth.

4. The method of claim 1, wherein the female subject is a pregnant human at 20 to 37 weeks of gestation.

5. The method of claim 4, wherein the pregnant human female subject is at risk of preterm birth.

6. The method of claim 5, wherein the pregnant human female subject at risk of preterm birth has a short cervical length, an infection, a placental anomaly, has had a prior cesarean delivery, has or had uterine fibroids, a connective tissue disorder, is pregnant from in vitro fertilization, diabetes, blood clotting problems, high blood pressure, vaginal bleeding, a personal history of preterm birth, a family history of preterm birth, a previous pregnancy with 18 months of the current pregnancy, a current multi-fetal pregnancy, a uterine abnormality, a cervical abnormality, is overweight before or during pregnancy, is underweight before or during pregnancy, or is carrying a fetus with known birth defects.

7. The method of claim 5, wherein the pregnant human female subject at risk of preterm birth is a human woman under the age of 20 or over the age of 35.

8. The method of claim 5, wherein the pregnant human female subject at risk of preterm birth is a human woman engaging in smoking, engaging in drinking alcohol, using illegal drugs, with limited or no healthcare during pregnancy, subject to stress, subject to long working hours with long periods of standing, or exposed to environmental pollutants.

9. The method of claim 1, further comprising checking the female subject for signs of preterm labor.

10. The method of claim 9, wherein checking the female subject for signs of preterm labor comprises a cervical exam, a transvaginal ultrasound exam, testing for amniotic fluid, or testing for fetal fibronectin.

11. The method of claim 1, wherein the exosomes that expresses IL-10 or that comprises IL-10 are not administered intraamnionically.

12. The method of claim 1, wherein the exosomes that expresses IL-10 or that comprises IL-10 are administered via a route that is oral, parenteral, intravenous, intra-arterial, subcutaneous, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, via inhalation, or combinations thereof.

13. A method of treating a pregnant female subject at risk of preterm birth, comprising administering to the pregnant female subject an effective amount of an exosome that comprises a nucleic acid that comprises IL-10 (eIL-10), wherein the effective amount of the eIL-10 is effective to reduce, prevent or delay preterm birth.

14. The method of claim 13, wherein preterm birth is spontaneous preterm birth.

15. The method of claim 13, wherein the pregnant human female presents to labor and delivery with symptoms of preterm birth.

16. The method of claim 13, wherein the female subject is a pregnant human at 20 to 37 weeks of gestation; wherein the pregnant human female subject is at risk of preterm birth; or wherein the pregnant human female subject at risk of preterm birth has a short cervical length, an infection, a placental anomaly, has had a prior cesarean delivery, has or had uterine fibroids, a connective tissue disorder, is pregnant from in vitro fertilization, diabetes, blood clotting problems, high blood pressure, vaginal bleeding, a personal history of preterm birth, a family history of preterm birth, a previous pregnancy with 18 months of the current pregnancy, a current multi-fetal pregnancy, a uterine abnormality, a cervical abnormality, is overweight before or during pregnancy, is underweight before or during pregnancy, or is carrying a fetus with known birth defects.

17. The method of claim 16, wherein the pregnant human female subject at risk of preterm birth is a human woman under the age of 20 or over the age of 35.

18. The method of claim 16, wherein the pregnant human female subject at risk of preterm birth is a human woman engaging in smoking, engaging in drinking alcohol, using illegal drugs, with limited or no healthcare during pregnancy, subject to stress, subject to long working hours with long periods of standing, or exposed to environmental pollutants.

19. The method of claim 13, further comprising checking the female subject for signs of preterm labor.

20. The method of claim 19, wherein checking the female subject for signs of preterm labor comprises a cervical exam, a transvaginal ultrasound exam, testing for amniotic fluid, or testing for fetal fibronectin.

21. The method of claim 13, wherein the exosomes that eIL-10 is not administered intraamnionically.

22. An exosome comprising a nucleic acid that expresses IL-10 or that comprises IL-10, wherein the exosomes are isolated for size and loading of the nucleic acid that expresses IL-10 or the IL-10.

23. The exosome of claim 22, wherein the exosomes are isolated from a media by at least one of: ultracentrifugation, purification, or size exclusion chromatography.

24. The exosome of claim 22, wherein the exosomes have a particle size ranging between 40 nm-110 nm.

25. The exosome of claim 22, wherein the nucleic acid that expresses IL-10 or the IL-10 are loaded into the exosomes by one or more electroporation steps.

26. The exosome of claim 22, wherein the exosomes are autologous.
